(19) United States
(12) Patent Application Publication
(10) Pub. No.: US 2007/0027447 A1
(43) Pub. Date: Feb. 1, 2007

(54) Seal for medical instrument

(75) Inventors: Marc Theroux, Grafton, MA (US); Michael Ewaschuk, Haverhill, MA (US); Henri DeGuillebon, Manchester, MA (US); Peter Aliski, Malden, MA (US); Dennis Pelletier, Salem, MA (US); Brian Mazejka, Salem, NH (US)

Correspondence Address: GREENBLUM & BERNSTEIN, P.L.C., 1950 ROLAND CLARKE PLACE, RESTON, VA, 20191, US

(73) Assignee: MICROLINE PENTAX INC., Beverly, MA (US)

(21) Appl. No.: 11/189,789

(22) Filed: Jul. 27, 2005

(51) Int. Cl. A61B 18/18

(52) U.S. Cl. 606/41

(57) ABSTRACT

A laparoscopic tube end for interfacing a tube with a tip may include an attachment portion to attach to the tip, and a tapered flare portion to frictionally abut the tip when the tip is attached to the tube end.

### 1. FIELD OF THE INVENTION

This invention relates to a medical instrument including an electrosurgical apparatus, such as a laparoscopic tube end used for performing laparoscopic, pelvoscopic, arthroscopic, thoroscopic and/or similar such procedures, and more particularly to an electrosurgical apparatus having a fluid-resistant seal for fluidically isolating and electrically insulating a detachable tip when engaged with a laparoscopic tube end.

### 2. BACKGROUND OF THE INVENTION

Medical procedures such as laparoscopy and the like, which employ a tip at the end of a tube for insertion into the patient, are beneficial because the incisions necessary to perform them are minimal in size, therefore promoting more rapid recovery and lower costs. For example, a patient who undergoes laparoscopic surgery may typically return to normal activity within a period of a few days to about a week, in contrast to more invasive procedures requiring a relatively larger incision (which may require about a month for recovery). (Although the term “laparoscopic” is typically used hereinafter, such use of the term “laparoscopic” should be understood to encompass any such similar or related procedures such as, for example, arthroscopic, endoscopic, pelvoscopic and/or thoroscopic or the like, in which relatively small incisions are used.)

However, when a tip is detachably connected to the tube end of a laparoscopic device, complications may occur if fluid breaches the connection and enters the interior of the tip or tube end. For example, septic contamination may arise in the laparoscopic device and/or electrical current may unintentionally leak therefrom.

### SUMMARY OF THE INVENTION

Accordingly, it is a feature of the present invention to mitigate the risk of electrical shock and/or contamination of a tube by fluids, and to provide isolation from electrical shock and/or shorting caused by fluid contact.

In view of the above-noted, and other, features, the present invention provides a seal for medical instruments for preventing electrical and/or fluidic contact between a tip and a tube end of a surgical or laparoscopic device.

According to an aspect of the present invention, a laparoscopic tube end may include a flange having a contact surface, a groove adjacent to the contact surface of the flange, and an engagement portion to engage with a tip, and a gasket which is elastically deformable, disposed in the groove to compressibly abut the contact surface of the flange when the tip is engaged with the engagement portion. The gasket may include an elastomeric material to electrically isolate an interior of the tube end from an exterior of the tube or tip when the tip compresses the gasket, and the attachment portion may include threading on an exterior surface of the laparoscopic tube end or an interior surface of the laparoscopic tube end. The laparoscopic tube end may include the tip, detachably engaged with the engagement portion. Also, the gasket may have a generally square, circular or rounded cross-section, and may be connected to the groove by an adhesive material or by the elasticity of the gasket. Further, the tip may compress the gasket against the contact surface of the flange and form a fluid-resistant seal between the tip and the tube end.

The laparoscopic tube end may also include an interior to receive a yoke of the tip, and an interior threaded portion to threadedly engage with the yoke of the tip. In addition, the gasket may have an outer radius which increases from an uncompressed radius to a maximal radius when the gasket is compressed by the tip against the contact surface of the flange, in which the maximal radius does not exceed an outermost radius of the flange. Further, grease may be disposed on any one or more of the flange, the groove, the engagement portion, the instrument tip or the gasket, and may seal and/or insulate an interior of the tube end from an exterior of the tube end; also, the engagement portion may include threading on an exterior surface of the instrument tip and/or an interior surface of the instrument tip.

In accordance with another aspect of the present invention, a tip for engaging with a laparoscopic tube end may include a threaded portion to threadedly engage with the laparoscopic tube end, a back hub connected to the threaded portion, and a seal which is elastically deformable and connected with the back hub, in which the seal may compressibly abut the laparoscopic tube end when the tip is engaged with the laparoscopic tube end. The tip may also include an extremity which abuts a flange of the laparoscopic tube end and the gasket of the laparoscopic tube end. Further, the tip may include a yoke (or, more generally, a stem, post or any other suitable internal member and the like, which may be generally equivalent with a yoke and for which the term “yoke” used hereinafter may be understood to encompass) which threadedly engages with an interior of the laparoscopic tube end, in which the resulting seal electrically insulates an external surface of the laparoscopic tube end or the tip from the interior of the laparoscopic tube end. Also, the seal may be molded to the back hub, or connected by adhesive.

In accordance with yet another aspect of the present invention, a laparoscopic tube end for interfacing a tube with a tip may include an attachment portion which threadedly attaches to the tip, the threaded portion including a semi-crystalline electrically resistive plastic material, and a tapered flare which may frictionally abut the tip when the tip is engaged with the tube end. The semi-crystalline electrically resistive plastic material may include at least one polyether ether ketone (PEEK) material, and the laparoscopic tube end may further include a groove in the attachment portion, and a band of heat-shrink material disposed in the groove. The heat-shrink material may include at least one fluorinated ethylene propylene (FEP) material and/or any other elastic or elastomeric material (such as, for example, plastic, rubber, etc., but not necessarily limited thereto) or combination thereof. The laparoscopic tube end may further include the tip engaged with the tube end, in which the attachment portion includes threading to detachably attach to the tip. The laparoscopic tube end may also include a metal interface fixedly attached to the tube end, and the metal interface may include stainless steel. Grease or any other such viscous fluid or material may also be disposed on the tip or tube end (or any components thereof) and may seal and/or insulate the interior of the tube end from the exterior of the tube end.

Other exemplary embodiments and advantages of the present invention may be ascertained by reviewing the present disclosure and the accompanying drawings.

### BRIEF DESCRIPTION OF THE DRAWINGS

The present invention is further described in the detailed description which follows, in reference to the noted drawings by way of non-limiting examples of certain embodiments of the present invention, in which the numerals represent like elements throughout the several views of the drawings, in which:

FIG. 1A illustrates an example of a laparoscopic medical device including a tip and a laparoscopic tube;

FIG. 1B illustrates a tip disengaged from a laparoscopic tube end, according to a first embodiment of the present invention;

FIG. 2A is a cutaway view illustrating a laparoscopic tube end having a gasket, according to the first embodiment;

FIG. 2B illustrates the laparoscopic tube end having the gasket, as shown in FIG. 2A, engaged with a back hub of a tip;

FIG. 3 is a partial cutaway view illustrating a back hub having threading;

FIG. 4 is a partial cutaway view illustrating a tip engaged with the laparoscopic tube end, according to a second embodiment of the present invention;

FIG. 5A illustrates the laparoscopic tube end according to the second embodiment having a seal with a square cross-section;

FIG. 5B illustrates the laparoscopic tube end according to the second embodiment hating a seal with a round cross-section;

FIG. 6A illustrates a laparoscopic tube end formed of PEEK material and a heat-shrink tubing, according to a third embodiment of the present invention; and

FIG. 6B is a cutaway view along line A-A shown in FIG. 6A, illustrating an interior of the laparoscopic tube end made of PEEK material and having the heat-shrink tubing and a tapered flare, as well as a stainless-steel tube end portion, according to the third embodiment.

### DETAILED DESCRIPTION OF THE PREFERRED EMBODIMENTS

The particulars shown herein are by way of example and for purposes of illustrative discussion of the embodiments of the present invention only, and are presented for providing what is believed to be the most useful and readily understood description of the principles and conceptual aspects of the present invention. In this regard, no attempt is made to show structural details of the present invention in more detail than is necessary for the fundamental understanding of the present invention, the description taken with the drawings making apparent to those skilled in the art how the several forms of the present invention may be embodied in practice.

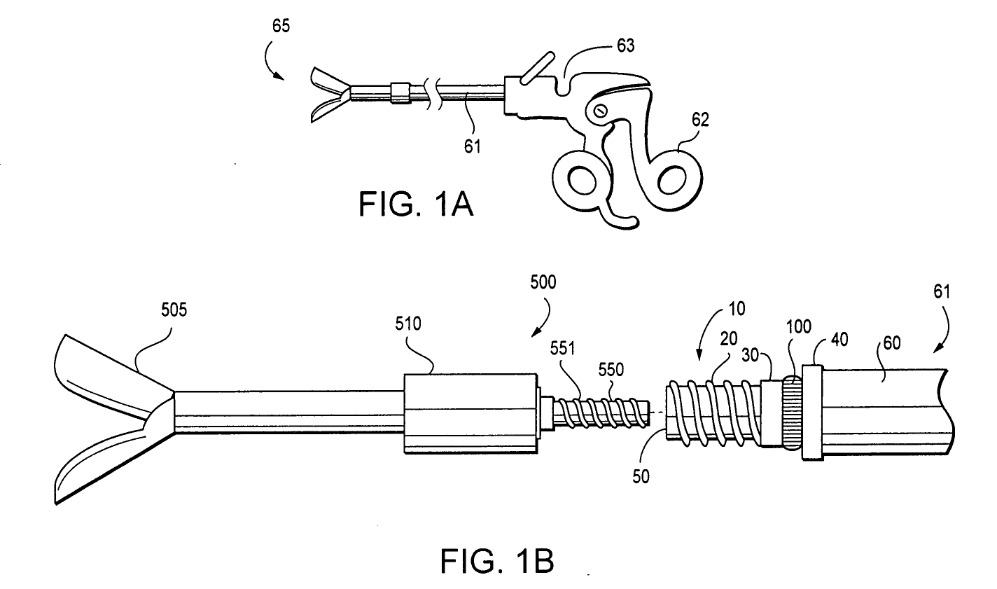

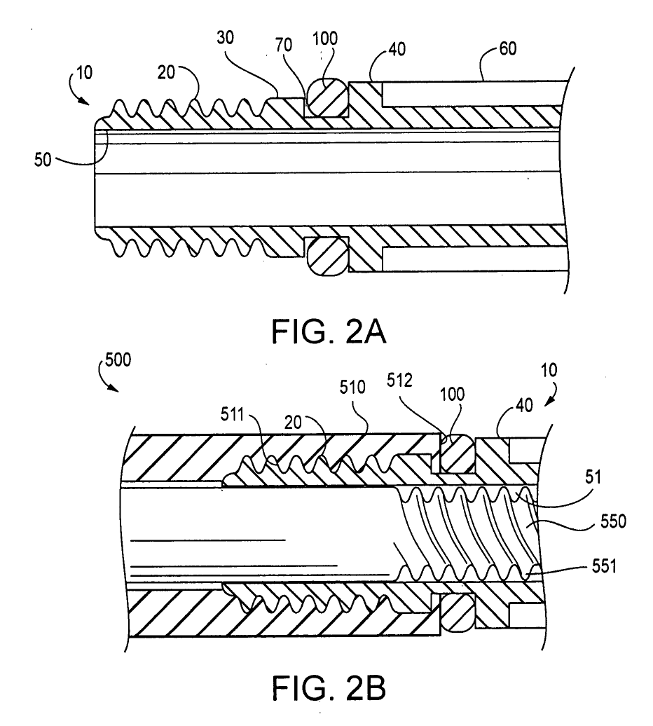

Referring to FIGS. 1A and 1B, a medical device 65 for performing laparoscopic surgery such as excision of tissue with cauterization, for example, may typically include (but is not limited to) a tip 500 having a tool 505 such as electrified shears or a cauterization tool, for example, in which the tip 500 may engage with the tube end 10 of a tube 61 which extends from a base portion 63 connected to handles 62 (or other suitable control device) operable by the medical personnel or physician performing the surgery. As shown in FIGS. 1B, 2A and 2B, for example, a laparoscopic tube end 10 according to a first embodiment of the present invention may include exterior threading 20 (which may threadedly engage and/or attach to a back hub 510 of the tip 500), as well as a flange 40 and outer sheath 60. Further, the laparoscopic tube end 10 according to the first embodiment may include a gasket 100 which may be either entirely or partially formed of an elastomeric material including, but not limited to, for example, natural or artificial rubber, plastic (such as, for example, polyethylene, polypropylene or any other suitable plastic polymer), and/or resin (and/or any suitable mixture or compound thereof, noting that the elastomeric material is not limited to the exemplary materials thus noted). Further, a gasket 100 may typically surround and conform to a groove 70 disposed between the exterior threading 20 and a flange 40 of the laparoscopic tube end, and the gasket 100 may have any suitable cross-section such as, for example, a generally square, round or rounded cross-section (see, for example, the generally rounded cross-section of the gasket 100 shown in FIG. 2A). The gasket 100 may be connected to the groove 70 by, for example, the elasticity inherent in the gasket 100 which is fitted around the exterior of the groove 70, and/or an adhesive, or by molding.

FIG. 2B shows the engagement of the back hub 510 of the tip 500 with the laparoscopic tube end 10 when the laparoscopic tube end 10 is fully engaged with the laparoscopic tube end 10. It is noted that although the back hub 510 and yoke 550 of the tip 500 are shown as engaging with respective portions of the laparoscopic tube end 10 via threading 511 and 551, respectively, the present invention is not limited to engagement by threading, but may alternatively use any suitable engagement technology such as, for example, a spring-loaded bead and socket mechanism, a barrel pin, or dog-tooth ratchet mechanism, among other suitable engagement mechanisms. Furthermore, the respective rates of the threading of the yoke 551 and the threading of the back hub 511 may be substantially different, although they may also be substantially equivalent in some implementations.

When the back hub 510 is fully engaged with the laparoscopic tube end 10 (by threading, for example), an abutment surface 512 of the back hub 510 may abut the gasket 100, and urge the gasket 100 against the flange 40. This engagement seals the interior of the laparoscopic tube end from any fluids surrounding the tip 500 or tube end 10, and electrically insulates and fluidically isolates the laparoscopic tube end 10 from the exterior of the tube 61 and tip 500 because the pressure between the abutment surface 512, gasket 100 and flange 40 and the adhesiveness and elasticity of the gasket 100 form a fluidic seal, and the electrically insulating properties of the elastomeric material of the gasket 100 form a high electrical impedance. In addition, the pressure of the back hub 510 when fully engaged against the gasket 100 may beneficially prevent rotation and/or disengagement of the back hub 510 from the laparoscopic tube end 10, because the frictional resistance resulting from the abutment of the abutment surface 512 of the back hub 510 against the elastomeric material of the gasket 100 tends to prevent rotation and unscrewing of the back hub 510 from the tube end 10. The composition, shape and/or materials of the gasket 100 may be selected to optimize the frictional contact with the back hub 510, the effectiveness of the fluidic seal, and/or the effectiveness of the electrical impedance thereof, for example.

Further, the height (or outermost radius) of the gasket 100, and/or the deformation properties of the gasket 100, may be selected such that the top surface of the gasket 100 does not exceed the height of the flange 40 when the back hub 510 is fully engaged with the laparoscopic tube end 10, for example, or such that the top of the gasket 100 deforms from an initial, uncompressed height to a maximal height thereof which forms a substantially continuous or smooth overall outer surface across the back hub 510, gasket 100 and flange 40, in order to minimize snags or unwanted friction during insertion or removal of the laparoscopic device 65, for example. Grease, oil, putty, epoxy, glue, resin, viscous fluid or material and/or any other suitable material (not shown) may also be used to seal and/or insulate the laparoscopic tube end 10 and/or other components, either alone or in combination with other ways of sealing and/or insulating.

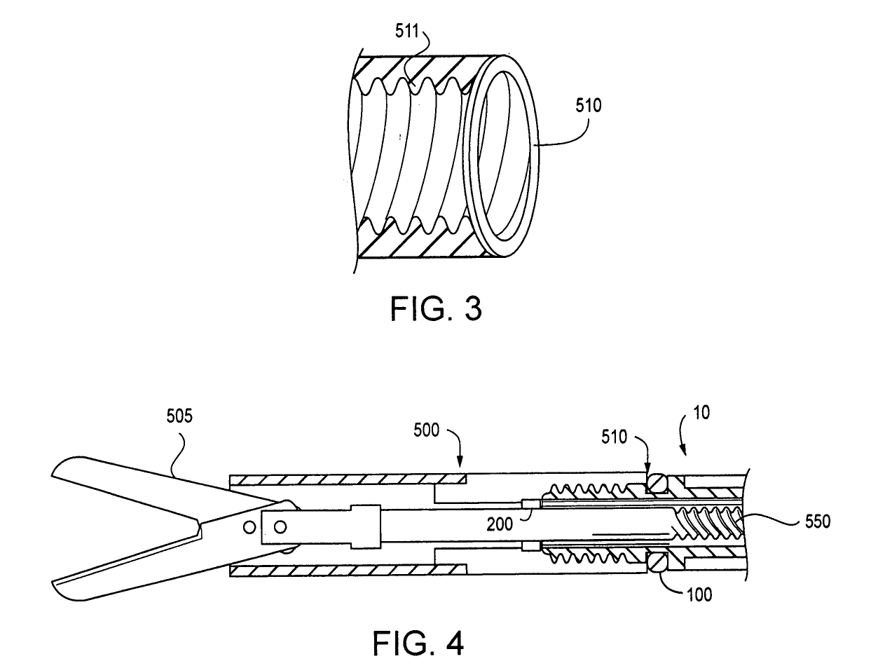

As shown in FIGS. 2A, 2B and 3, for example, the laparoscopic tube end 10 may include an interior 50, having interior threading 51 for engaging with the threading 551 of the yoke 550 of the tip 500. Further, as shown in FIG. 3, the back hub 510 of the tip 500 may also include threading 511 for engaging with the exterior threading 20 of the tube end 10, and the respective threading pitches of the threading of the yoke 551 and the threading of the back hub 511 may be different.

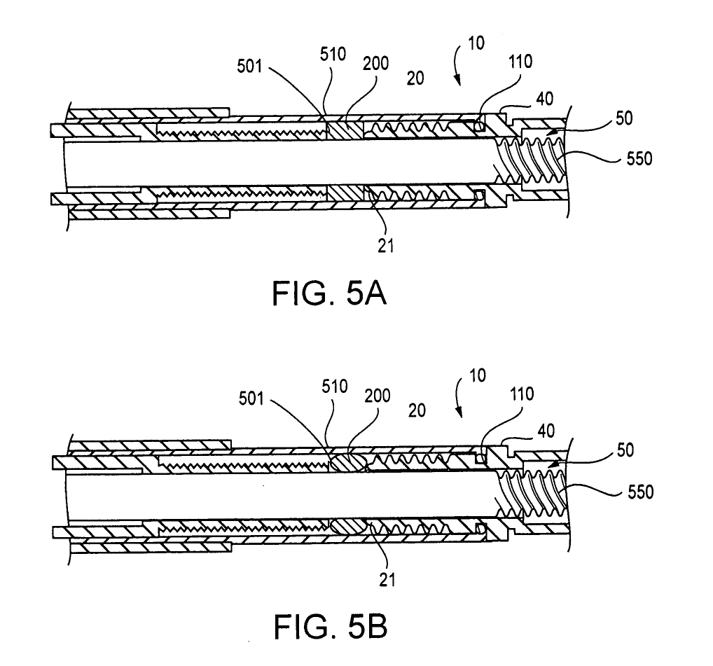

FIGS. 4, 5A and 5B illustrate a second embodiment of the present invention, in which the tip 500. includes a first seal 200 where the back hub 510 contacts an extremity of the tube end 10. Accordingly, as the back hub 510 is tightened against the tube end 10, a seal is created by compression of the elastomeric material of the first seal 200 between the distal tip 21 of the tube end 10 and the compression surface 501 of the back hub 510, for example. Although the first seal 200 is exemplified as attached to the back hub 510 of the tip 500, it may alternatively be attached to the extremity of the tube end 10, for example. Furthermore, the cross-section of the first seal 200 may have any appropriate form, such as square, circular, or any other suitable form.

As shown in FIG. 5A, the tube end 10 may also include a second seal 110 adjacent to the flange 40 of the laparoscopic tube end 10. According to one aspect of the second embodiment, the second seal 110 may have a cross-sectional diameter or area which is smaller than that of the first seal 200 (which may be round as shown in FIG. 5B or rectangular as shown in FIG. 5A, for example), permitting enhanced sealing and insulating effectiveness. According to yet another aspect of the second embodiment, the second seal 110 may have a size and form suitable for allowing the back hub 510 to abut flushly against the flange 40 of the laparoscopic tube end 10, while forming a seal generated by compressive contact of the bottom surface of the back hub 510 against the elastomeric material of the second seal 110 and the floor of the groove 70.

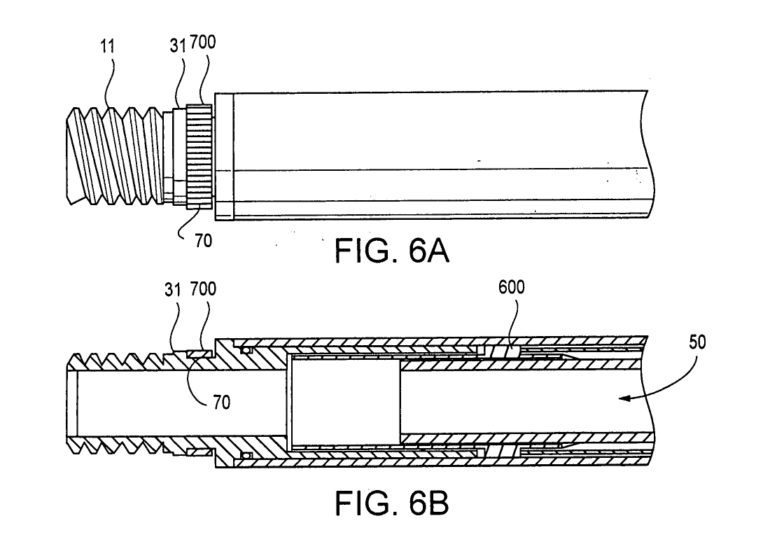

FIGS. 6A and 6B illustrate a third embodiment of the present invention, in which a laparoscopic device includes a laparoscopic tube end 11 made either entirely or at least partially of a semi-crystalline electrically-resistive plastic material (such as, for example, polyether ether ketone, or PEEK). According to another aspect of the third embodiment, the laparoscopic tube end 11 may include a flare 31 or tapered section to provide at least a slight interference fit with the back hub of the tip. Friction resulting from the interference fit of the flare 31 and back hub provides an anti-torque effect, enhancing the stability of the tip for staying in place while the tip is engaged with the laparoscopic tube end 11 during use of the laparoscopic device 65.

According to another aspect of the third embodiment, the laparoscopic tube end 11 may include a groove 70 into which a band 700 of heat-shrink material is disposed. The heat-shrink material, which may be formed either entirely or partially of at least one type of fluorinated ethylene propylene (FEP) material, may have a shape and material composition selected so as to generate minimal additional friction during engagement of the tip 500 to the laparoscopic tube end 11 beyond the friction that would otherwise produced by the laparoscopic tube end 11 if the band 700 of heat-shrink material were not included. When the tip 500 is engaged with the laparoscopic tube end 11, the band 700 of heat-shrink material may provide a water-resistant seal, enhancing the fluid-resistant and electrically insulating properties of the laparoscopic device 65.

In a further aspect of the third embodiment, the interior 50 of the tube end 11 may include a metal interface or adapter 600 made of stainless steel or any other suitable material (such as a biologically inert ferrous or non-ferrous metal, for example) for use in medical devices, in which the metal interface or adapter 600 is bonded to the interior 50 of the tube end 11 (by molding, adhesives, or welding, for example). The metal interface 600 may provide a surface suitable for welding to other components, and may include a sufficiently durable material for incorporating a slot mechanism or movement assembly such as, for example, a slot feature and/or barrel pin which controls the movement of an inner shaft assembly.

Because a laparoscopic tube end 11 that is composed of a semi-crystalline electrically resistive plastic material such as PEEK provides electrical resistivity, especially in conjunction with a band 700 of heat-shrink material composed of, for example, FEP to create a fluid resistive seal, the overall electrically insulating and fluidically isolating qualities of the laparoscopic device 65 may be enhanced.

In addition, although the laparoscopic device 65 has been exemplified as including a tool 505 in the form of shears on a tip 500, it is to be understood that the present invention is not limited thereto but may alternatively include, for example, a grasping tool, an optical device and/or light, electrical or heat cauterization tool, or any other tool suitable for use in a laparoscopic or other surgical or medical device.

It is noted that the foregoing examples have been provided merely for the purpose of explanation and are in no way to be construed as limiting of the present invention. While the present invention has been described with reference to certain embodiments, it is understood that the words which have been used herein are words of description and illustration, rather then words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed:

1. A laparoscopic tube end for interfacing a tube with an instrument tip, comprising:
a flange including a contact surface;
a groove adjacent to the contact surface of the flange;
an engagement portion configured to engage with the tip; and
a gasket which is elastically deformable, disposed in the groove and configured to compressibly abut the contact surface of the flange when the tip is attached to the engagement portion.

2. The laparoscopic tube end according to claim 1, wherein the gasket includes an elastomeric material configured to electrically isolate an interior of the tube end from an exterior of the tube end when the tip compresses the gasket.

3. The laparoscopic tube end according to claim 1, wherein the engagement portion includes threading on at least one of an exterior surface of the laparoscopic tube end or an interior surface of the laparoscopic tube end.

4. The laparoscopic tube end according to claim 1, wherein the tip is detachably engagable with the engagement portion.

5. The laparoscopic tube end according to claim 1, wherein the gasket has one of a generally square, circular or rounded cross-section, and is connected to the groove by an adhesive material.

6. The laparoscopic tube end according to claim 1, wherein the tip is configured to compress the gasket against the contact surface of the flange and form a fluid-resistant seal between the tip and the tube end.

7. The laparoscopic tube end according to claim 1, further comprising:
an interior configured to receive a yoke of the tip; and
an interior threaded portion configured to threadedly engage with the yoke of the tip.

8. The laparoscopic tube end according to claim 1, wherein the gasket has an outer radius configured to increase from an uncompressed radius to a maximal radius when the gasket is compressed by the tip against the first surface of the flange, and wherein the maximal radius does not exceed a radius of the flange.

9. The laparoscopic tube end according to claim 1, further comprising grease disposed on at least one of the flange, the groove, the engagement portion, the instrument tip or the gasket and configured to at least one of seal or insulate an interior of the tube end from an exterior of the tube end.

10. The laparoscopic tube end according to claim 1, wherein the engagement portion includes threading on at least one of an exterior surface of the instrument tip or an interior surface of the instrument tip.

11. A tip for engaging a laparoscopic tube end, comprising:
a threaded portion configured to threadedly engage with a laparoscopic tube end;
a back hub connected with the threaded portion; and
a seal which is elastically deformable and connected with the back hub, the seal configured to compressibly abut the laparoscopic tube end when the tip is engaged with the laparoscopic tube end.

12. The tip according to claim 11, further comprising an extremity configured to abut a flange of the laparoscopic tube end and a gasket of the laparoscopic tube end.

13. The tip according to claim 11, further comprising a yoke configured to threadedly engage an interior of the laparoscopic tube end, wherein the seal electrically insulates an external surface of the laparoscopic tube end from the interior of the laparoscopic tube end.

14. The tip according to claim 11, wherein the seal is molded to the back hub.

15. The tip according to claim 11, further comprising grease disposed on at least one of the threaded portion, the laparoscopic tube end, the back hub, or the seal and configured to at least one of seal or insulate an interior of the laparoscopic tube end from an exterior of the laparoscopic tube end.

16. A laparoscopic tube end for interfacing a tube with a tip, comprising:
an attachment portion configured to attach to the tip, the attachment portion including a semi-crystalline electrically resistive plastic material; and
a tapered flare portion configured to frictionally abut the tip when the tip is attached to the tube end.

17. The laparoscopic tube end according to claim 16, wherein the semi-crystalline electrically resistive plastic material includes at least one polyether ether ketone material.

18. The laparoscopic tube end according to claim 16, further comprising:
a groove in the attachment portion; and
a band of heat-shrink material disposed in the groove.

19. The laparoscopic tube end according to claim 18, wherein the heat-shrink material includes at least one fluorinated ethylene propylene material.

20. The laparoscopic tube end according to claim 16, further comprising the tip frictionally abutting the tapered flare portion.

21. The laparoscopic tube end according to claim 16, wherein the attachment portion includes threading configured to detachably engage with the tip.

22. The laparoscopic tube end according to claim 16, further comprising a metal interface fixedly attached to the tube end.

23. The laparoscopic tube end according to claim 21, wherein the metal interface includes stainless steel.

24. The tip according to claim 16, further comprising grease disposed on at least one of the attachment portion, the tip, the laparoscopic or the tapered flare and configured to at least one of seal or insulate an interior of the laparoscopic tube end from an exterior of the laparoscopic tube end.
